(19) United States
(12) Patent Application Publication
(10) Pub. No.: US 2021/0155651 A1
(43) Pub. Date: May 27, 2021

(54) PROCESS FOR THE PREPARATION OF IRON (III) CARBOXYMALTOSE

(71) Applicants: Thirumalai Rajan Srinivasan, Sangareddy (Dist), Hyderabad, Telangana (IN); MSN LABORATORIES PRIVATE LIMITED, R&D CENTER, Sangareddy (Dist),Hyderabad, Telangana (IN)

(72) Inventors: Thirumalai Rajan SRINIVASAN, Hyderabad (IN); Eswaraiah SAJJA, Hyderabad (IN); Venkata Panakala Rao GOGULAPATI, Hyderabad (IN); Ganapathi Chary NAGUNURI, Hyderabad (IN); Mohammad Rafee SHAIK, Hyderanad (IN)

(73) Assignee: MSN Laboratories Private Limited, R&D Center, Sangareddy (Dist), Hyderabad, Telangana (IN)

(21) Appl. No.: 17/044,310

(22) PCT Filed: Apr. 4, 2019
(86) PCT No.: PCT/IN2019/050280

§ 371 Date: Sep. 30, 2020

(30) Foreign Application Priority Data
Apr. 5, 2018 (IN) 201841012945

(51) Int. Cl. C07H 3/06; A61K 9/00

(52) U.S. Cl. CPC C07H 3/06; A61K 9/0019

(57) ABSTRACT

Water soluble iron carbohydrate complex obtainable from an aqueous solution of iron (III) salt and an aqueous solution of the oxidation product of one or more maltrodextrins using an aqueous oxone solution at a pH-value within the alkaline range, where, when one maltodextrin is present, its dextrose equivalent lies between 5 and 20, and when a mixture of several maltodextrins is present, the dextrose equivalent of the mixture lies between 5 and 20 and the dextrose equivalent of each individual maltodextrin contained in the mixture lies between 2 and 40, process for its production and medicament for the treatment and prophylaxis of iron deficiency anaemia conditions.

### RELATED APPLICATIONS

This application claims the benefit of priority of our Indian patent application number 201841012945 filed on 05 Apr. 2018 which are incorporated herein by reference.

### FIELD OF THE INVENTION

The present invention relates an improved process for the preparation of iron [III] carboxymaltose.

### BACKGROUND OF THE INVENTION

Iron deficiency anemia is a common hematological complication with potentially serious clinical consequences that may require intravenous iron therapy.

Ferric carboxymaltose, an iron replacement product, is an iron carbohydrate complex with the chemical name of polynuclear iron (III) hydroxide 4(R)-(poly-(1→4)-O-α-D-gluco pyranosyl)-oxy-2(R),3(S),5(R),6-tetrahydroxy-hexanoate. The relative molecular weight is approximately 150 000 Da.

Ferric carboxymaltose is a stable, non-dextran iron formulation administrated intravenously in large single doses to treat iron deficiency anemia. It is an iron complex that consists of a ferric hydroxide core stabilized by a carbohydrate shell. Ferric carboxymaltose complex, also known as iron (III) carboxymaltose, is indicated for the treatment of iron deficiency anemia, and which is marketed by Vifor under the brand name Ferinject.

As per Australian Public Assessment Report (AUSPAR) published in the year 2011, the oxidation process does not appear to be a reaction which will necessarily give a well-defined polymer. The chemical nature of a batch of ferric carboxymaltose is thus dependent on the consistency of control of the oxidation process among other variables.

U.S. Pat. No. 7612109 B2 herein after referred as “US'109” discloses Water-soluble iron carbohydrate complexes (ferric carboxymaltose complexes) obtainable from an aqueous solution of an iron (III) salt, preferably iron (III) chloride, and an aqueous solution of the oxidation product of one or more maltodextrins using an aqueous hypochlorite solution.

US2012/0214986 A1 discloses process for the preparation of ferric carboxymaltose comprising oxidation of maltodextrins using an aqueous sodium hypochlorite solution and subsequently reacting the obtained oxidized maltodextrin with ferric hydroxide to produce ferric carboxymaltose with inconsistence in the molecular weight.

US2018/0105609 A1 discloses process for the preparation of ferric carboxymaltose comprising oxidation of maltodextrins using organic hypo halite in the presence of catalyst such as transition metal catalyst and phase transfer catalyst and subsequently reacting the obtained oxidized maltodextrin with ferric hydroxide to produce ferric carboxymaltose with inconsistence in the molecular weight. The said process also involves usage of expensive reagents and also additional reagents in the process. This results in the increase in the production cost and hence making the process uneconomical and not suggestible for commercial scale.

The process for the preparation of ferric carboxymaltose complex described in the aforementioned prior art suffers from disadvantages since it involves the use of excess amounts of sodium carbonate and iron (III) chloride, thereby generating a large quantity of chemical waste which is difficult to treat. Moreover, the use of highly corrosive and toxic reagents like iron (III) chloride and excess amounts of sodium carbonate in the final stage of complex formation is not advisable since the resulting complex is contaminated with unacceptable amounts of impurities like sodium carbonate and sodium chloride, and thus resulting in a poor product quality.

IN3463/MUM/2011 discloses a process for the preparation of ferric carboxymaltose comprising oxidation of maltodextrin with sodium hypochlorite to provide oxidized maltodextrin followed by reacting with iron (III) salt to provide ferric carboxymaltose. The said process involves addition of sodium hypochlorite at higher temperatures i.e. at 65-70° C. which is not suggestible. As sodium hypochlorite explodes on heating and hence the said process is not recommendable for commercial scale.

WO2016151367 A1 describes a process for preparation of iron (III) carboxymaltose complex which include reacting aqueous solution of iron (III) salt with aqueous solution of oxidation product of at least one maltodextrin. The oxidation of maltodextrin is carried out in presence of catalyst and phase transfer catalyst in alkaline medium. The processes in the art require catalysts, phase transfer catalysts for performing oxidation of maltodextrin which add to the manufacturing cost. The use of sodium hypochlorite as the oxidizing agent lead to formation of undesired chlorinated by products. The processes in the art thus suffer from the drawbacks of formation of inorganic impurities such as metal bromides, chlorides and carbonates which impact the yield and purity of iron (III) carboxymaltose.

Based on the aforementioned drawbacks, the prior art process have been found to be unsuitable for the preparation of iron (III) carboxymaltose at lab scale and on commercial scale.

Hence, there is an unmet need to find an efficient and industrially advantageous process for the preparation of iron (III) carboxymaltose which is commercially viable and which overcomes the problems associated with the prior art. The present invention involves oxidation of maltodextrin using simple, cheaper, non-toxic and versatile oxidizing agent like oxone for the oxidation of maltodextin and converting the oxidized maltodextrin to Iron (III) carboxymaltose.

### BRIEF DESCRIPTION OF THE INVENTION

First embodiment of the present invention provides a process for the preparation of oxidized maltodextrins.

Second embodiment of the present invention provides an improved process for the preparation of water soluble iron (III) carboxymaltose.

### DETAILED DESCRIPTION OF THE INVENTION

As used herein the term “suitable solvent” used in the present invention refers to “hydrocarbon solvents” such as n-hexane, n-heptane, cyclohexane, pet ether, toluene, pentane, cycloheptane, methyl cyclohexane, m-, o-, or p-xylene, and the like; “ether solvents” such as dimethoxy methane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, 1,2-dimethoxy ethane and the like; “ester solvents” such as methyl acetate, ethyl acetate, isopropyl acetate, n-butyl acetate and the like; “polar-aprotic solvents such as dimethylacetamide (DMA), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcoholic solvents” such as methanol, ethanol, n-propanol, isopropanol, n-butanol, isobutanol, t-butanol, 2-nitroethanol, 2-fluoroethanol, 2,2,2-trifluoroethanol, ethylene glycol, 2-methoxyethanol, 1, 2-ethoxyethanol, diethylene glycol, 1, 2, or 3-pentanol, neo-pentyl alcohol, t-pentyl alcohol, diethylene glycol monoethylether, cyclohexanol, benzyl alcohol, phenol, or glycerol and the like; “polar solvents” such as water or mixtures thereof.

The term “suitable base” used herein the present invention is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; “alkali metal alkoxides” such as sodium methoxide, sodium ethoxide, sodium tert-butoxide, potassium methoxide, potassium ethoxide, potassium tert-butoxide and the like; ammonia; and organic bases such as triethyl amine, methyl amine, ethyl amine, ethylenediamine.

As used herein the term “dia-filtering” in the present invention refers to a dilution process that involves removal or separation of components (permeable molecules like salts, small proteins, solvents etc.,) of a solution based on their molecular size by using micro-molecule permeable filters in order to obtain pure solution.

As used herein the term “dispersity index” (DI), or “formerly polydispersity index” (PDI), or “polydispersity” (PD) in the present invention refers to measure of the distribution of molecular mass in a given polymer sample.

As used herein the term Ferric carboxymaltose of the present invention can be also referred to as water soluble iron (III) carboxymaltose or iron (III) carboxymaltose.

In the first embodiment, the present invention provides a process for the preparation of oxidized maltodextrin, comprising oxidizing one or more maltodextrins with a suitable oxidizing agent at alkaline pH in the presence or absence of a suitable catalyst to provide oxidized maltodextrins,

In an embodiment of the present invention the suitable oxidizing agent is oxone.

In an embodiment of the present invention, when one maltodextrins is present, the maltodextrins has a dextrose equivalent (DE) of between 5 and 20, and wherein, when a mixture of more than one maltodextrins are present, the dextrose equivalent of each individual maltodextrins is between 2 and 40, and the dextrose equivalent of the mixture is between 5 and 20.

In an embodiment of the present invention, the oxidation is carried out in an alkaline solution, e.g. at a pH of about 8 to 12. As an example, oxidation can be carried out at temperatures in the order of about 15° C. to about 40° C., preferably of about 25° C. to about 35° C. The reaction times are, e.g. in the order of 10 minutes to 4 hours.

In an embodiment of the present invention, oxidation of one or more maltodextrin is catalyzed by adding of bromide ions, e.g. in the form of alkali bromides, for example sodium bromide. The added amount of sodium bromide is not critical and the amount is kept as low as possible catalytic amount in order to achieve an end product.

In an embodiment the present invention provides a process for the preparation of iron (III) carboxymaltose comprising oxidizing one or maltodextrins using oxone.

The second embodiment of the present invention provides an improved process for the preparation of water soluble iron (III) carboxymaltose, comprising:

a) Oxidizing one or more maltodextrins with a suitable oxidizing agent at alkaline pH and in the presence or absence of a suitable catalyst to provide oxidized maltodextrins,
b) reacting the oxidized maltodextrins with suitable iron (III) salt to provide water soluble iron (III) carboxymaltose.

In an embodiment of the present invention, when one maltodextrins is present, the maltodextrin has a dextrose equivalent (DE) of between 5 and 20, and wherein, when a mixture of more than one maltodextrins is present, the dextrose equivalent of each individual maltodextrins is between 2 and 40, and the dextrose equivalent of the mixture is between 5 and 20.

In an embodiment of the present invention, the suitable oxidizing agent is oxone.

In an embodiment of the present invention the mole ratio of oxone used is ranging from 1.0 to 5.0 moles per 1 mole of Maltodextrin.

In an embodiment of the present invention, oxidation is carried out in an alkaline solution, e.g. at a pH of about 8 to 12. Oxidation can be carried out at temperatures in the order of about 15° C. to about 40° C., preferably of about 25° C. to about 35° C. The reaction times are in the order of 10 minutes to 4 hours.

In an embodiment of the present invention, the oxidized maltodextrins can be isolated and dissolved; however, it is also possible to use the obtained aqueous solutions of the oxidized maltodextrins directly for the further reaction with the aqueous iron (III) solutions.

In an embodiment of the present invention treating oxidized maltodextrins with an iron (III) salt in an aqueous solution to provide water soluble iron (III) carboxymaltose.

In an embodiment of the present invention, water soluble salts of inorganic or organic acids, or mixtures thereof, such as halides, e.g. chloride and bromide or sulfates can be used as iron (III) salts. It is especially preferred to use an aqueous solution of iron (III) chloride.

In an embodiment of the present invention, the aqueous solution of the oxidized maltodextrins can be mixed with an aqueous solution of the Iron (III) salt to carry out the reaction.

In an embodiment of the present invention, after mixing of the oxidized maltodextrins and the iron (III) salt the pH of the reaction mixture is raised to values of at least 5, for example up to 11, 12, 13 or 14.

In an embodiment of the present invention, the pH of the reaction mixture is preferably raised slowly or gradually, for example, can be achieved by first adding a weak base, for example, up to a pH of about 3, and then neutralizing further using a stronger base.

In an embodiment of the present invention, examples of weak bases are alkali or alkaline earth-carbonates, bicarbonates, such as sodium and potassium carbonate or bicarbonate, or ammonia. Examples of strong bases are alkali or alkaline earth-hydroxides such as sodium, potassium, calcium or magnesium hydroxide.

In embodiment of the present invention, heating the reaction mixture to a temperature ranging from 30° C. to 100° C.

In an embodiment of the present invention, after completion of the reaction, the obtained solution can be cooled to e.g. room temperature and can optionally be diluted and optionally be filtered. After cooling, the pH can be adjusted to the neutral point or a little below, for example, to values of about 5 to 7, by the addition of an acid or base. It is possible to use e.g. the acids and bases which have been mentioned for carrying out the reaction. The solutions obtained are purified and can directly be used for the production of medicaments.

In an embodiment of the present invention, it is also possible to isolate the iron (III) complexes from the solution e.g. by precipitation with a ketone solvent, ester solvent or alcohol solvent.

In an embodiment of the present invention, isolation can also be effected by spray-drying. Purification can take place in the usual way, especially in order to remove salts.

The iron content of the obtained iron (III) carbohydrate complexes is 10 to 40% weight/weight, especially 20 to 35% weight/weight.

Prior art processes involve process for the preparation of iron (III) carboxymaltose comprising oxidizing maltodextrin using sodium hypochlorite. Sodium hypochlorite is highly unstable and decomposes on heating and requires additional safety precautions. Further, different lots of sodium hypochlorite have inconsistent assay which may lead to inconsistency in the molecular weight ranges of oxidized maltodextrins in turn ferric carboxymaltose.

The present invention involves oxidation of maltodextrin using oxone which is a solid with high stability and can be easily handled on commercial scale. Moreover using oxone the inventors of the present invention were able to get consistence in the molecular weight of iron (III) carboxymaltose.

Prior known processes involve process for the preparation of iron (III) carboxymaltose comprising treating maltodextrin with a suitable oxidizing agent to provide oxidized maltodextrin which upon directly treating with iron (III) salt without any purification or dia filtration provided iron (III) carboxymaltose with inconsistency in molecular weight and polydispersity. The inconsistency in the specification of iron (III) carboxymaltose is due to the presence of unwanted byproducts, low molecular weight oxidized maltodextrins and also residual salts present in the crude oxidized maltodextrins.

In one variant, inventors of the present invention have overcome the problem associated with the prior art by dia-filtering the oxidized maltodextrin to remove the unwanted byproducts, low molecular weight oxidized maltodextrins and also residual salts to provide pure oxidized maltodextrins.

In an aspect, the oxidized maltodextrins can be isolated in the form of a solid by conventional techniques known to a person skilled in the art and converts it into iron (III) carboxymaltose.

The starting material maltodextrin used in the present invention is commercially available.

Iron (III) carboxymaltose of the present invention form aqueous medicaments which are particularly suitable for parenteral, particularly intravenous, and also intramuscular administration, as well as for oral or topical administration, and can be used in particular for the treatment of iron deficiency anemia. Another object of the present invention therefore also relates to the use of the iron (III) carboxymaltose according to the present invention for the treatment and prophylaxis of iron deficiency anemia or for the production of medicaments for the parenteral treatment of iron deficiency anemia.

The preparation of the parenterally administrable solutions can be carried out in a customary manner, if appropriate with concomitant use of additives customary for parenteral solutions. The solutions can be formulated so that they can be administered as such by injection or as an infusion, eg in saline. For oral or topical administration, preparations may be formulated with appropriate conventional excipients and adjuvants.

The process described in the present invention was demonstrated in examples illustrated below. These examples are provided as illustration only and therefore should not be construed as limitation of the scope of the invention.

### EXAMPLES

### Example-1: Preparation of Iron (III) Carboxymaltose

### Step-a)

Water (50 ml) was added to maltodextrin (25 gms) at 25-30° C. and stirred for 45 minutes at the same temperature. Sodium bromide (0.17 gms) was added to the reaction mixture at 25-30° C. Aqueous sodium hydroxide solution was added to the reaction mixture at 25-30° C. Aqueous Oxone solution was added to the reaction mixture at 25-30° C. and stirred for 45 minutes at the same temperature. Methanol (150 ml) was slowly added to the reaction mixture at 25-30° C. and stirred for 45 minutes at the same temperature. Filtered the precipitated solid and dried to get the title compound. Yield: 13.0 gms.

### Step-b)

Water (26.0 ml) was added to oxidized maltodextrin (13.0 gms) at 25-30° C. and stirred for 45 minutes at the same temperature. Ferric chloride (11.0 gms) was slowly added to a pre-cooled water (26.0 ml) at 10-15° C. and stirred for 15 minutes at the same temperature. Slowly added the ferric chloride solution to the reaction mixture at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 40 minutes at the same temperature. Heated the reaction mixture to 95-100° C. and stirred for 40 minutes at the same temperature. Cooled the reaction mixture to 25-30° C. Filtered the reaction mixture through hyflow bed. The obtained filtrate was purified by dia-filtration technique. Acetone (260 ml) was added to the obtained compound at 25-30° C. and stirred for 1 hour at the same temperature. Filtered the precipitated solid, washed with acetone and dried to get the title compound. Yield: 12.5 gms.

Molecular Wt.: 151 kDa; Iron Content: 29.11%; Polydispersity index (PDI): 1.30

### Example-2: Preparation of Iron (III) Carboxymaltose

Water (100 ml) was added to maltodextrin (50 gms) at 25-30° C. and stirred for 45 minutes at the same temperature. Sodium bromide (0.33 gms) was added to the reaction mixture at 25-30° C. Aqueous sodium hydroxide solution was added to the reaction mixture at 25-30° C. Aqueous Oxone solution was slowly added to the reaction mixture at 25-35° C. and stirred the reaction mixture for 45 minutes at the same temperature. Aqueous ferric chloride solution was slowly added to the reaction mixture at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 40 minutes at the same temperature. Heated the reaction mixture to 95-100° C. and stirred for 40 minutes at the same temperature. Cooled the reaction mixture to 25-30° C. Filtered the reaction mixture through hyflow bed and washed the bed with water. Further, purified the obtained filtrate by dia-filtration. Adjusted the pH of the obtained filtrate using aqueous hydrochloric acid solution. Aqueous sodium chloride solution was added to the reaction mixture at 25-30° C. Acetone (1.1 lts) was added to the reaction mixture at 25-30° C. and stirred for 1 hour at the same temperature. Filtered the precipitated solid, washed with acetone and dried to get the title compound. Yield: 45.0 gms.

Iron Content: 29.14%; Molecular Wt.: 150 kDa; Polydispersity index (PDI): 1.28.

### Example-3: Preparation of Iron (III) Carboxymaltose

Water (1000 ml) was added to maltodextrin (500 gms) at 25-30° C. and stirred for 45 minutes at the same temperature. Sodium bromide (3.3 gms) was added to the reaction mixture at 25-30° C. Aqueous sodium hydroxide solution was added to the reaction mixture at 25-30° C. Aqueous Oxone solution was slowly added to the reaction mixture at 25-35° C. and stirred the reaction mixture for 45 minutes at the same temperature. Aqueous ferric chloride solution was slowly added to the filtrate at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 30 minutes at the same temperature. Heated the reaction mixture to 95-100° C. and stirred for 30 minutes at the same temperature. Cooled the reaction mixture to 25-30° C. Filtered the reaction mixture through hyflow bed and washed the bed with water. Further, purified the obtained filtrate by dia-filtration. Adjusted the pH of the obtained filtrate using aqueous sodium hydroxide solution. Aqueous sodium chloride solution was added to the reaction mixture at 25-30° C. Acetone (2.0 lts) was added to the reaction mixture at 25-30° C. and stirred for 1 hour at the same temperature. Filtered the precipitated solid, washed with acetone and dried to get the title compound. Yield: 200 gms.

Iron Content: 30.24%; Molecular Wt.: 152 kDa; Polydispersity index (PDI): 1.44.

### Example-4: Preparation of Iron (III) Carboxymaltose

Water (200 ml) was added to maltodextrin (100 gms) at 25-30° C. and stirred for 45 minutes at the same temperature. Sodium bromide (0.675 gms) was added to the reaction mixture at 25-30° C. Aqueous sodium hydroxide solution was added to the reaction mixture at 25-30° C. Aqueous Oxone solution and as well as aqueous sodium hydroxide solution was slowly added to the reaction mixture at 25-35° C. and stirred the reaction mixture for 45 minutes at the same temperature. Aqueous ferric chloride solution was slowly added to the filtrate at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 30 minutes at the same temperature. Heated the reaction mixture to 95-100° C. and stirred for 30 minutes at the same temperature. Cooled the reaction mixture to 25-30° C. Filtered the reaction mixture through hyflow bed and washed the bed with water. Further, purified the obtained filtrate by dia-filtration. Adjusted the pH of the obtained filtrate using aqueous sodium hydroxide solution. Aqueous sodium chloride solution was added to the reaction mixture at 25-30° C. Acetone (500 ml) was added to the reaction mixture at 25-30° C. and stirred for 1 hour at the same temperature. Filtered the precipitated solid, washed with acetone and dried to get the title compound.

Yield: 62.0 gms; Iron Content: 30.79%; Molecular Wt.: 151 kDa; Polydispersity: 1.27.

### Reference Example-1: Preparation of Iron (III) Carboxymaltose

Water (1000 ml) was added to maltodextrin (500 gms) at 25-30° C. and stirred for 45 minutes at the same temperature. Sodium bromide (3.3 gms) was added to the reaction mixture at 25-30° C. Aqueous sodium hydroxide solution was added to the reaction mixture at 25-30° C. Aqueous sodium hypochlorite solution was slowly added to the reaction mixture at 25-35° C. and stirred the reaction mixture for 45 minutes at the same temperature. Aqueous ferric chloride solution was slowly added to the filtrate at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 30 minutes at the same temperature. Heated the reaction mixture to 95-100° C. and stirred for 30 minutes at the same temperature. Cooled the reaction mixture to 25-30° C. Filtered the reaction mixture through hyflow bed and washed the bed with water. Further, purified the obtained filtrate by dia-filtration. Adjusted the pH of the obtained filtrate using aqueous sodium hydroxide solution. Aqueous sodium chloride solution was added to the reaction mixture at 25-30° C. Acetone (4.0 lts) was added to the reaction mixture at 25-30° C. and stirred for 1 hour at the same temperature. Filtered the precipitated solid, washed with acetone and dried to get the title compound.

Yield: 400 gms; Iron Content: 30.24%; Molecular Wt.: 134 kDa.

### Reference Example-2: Preparation of Iron (III) Carboxymaltose

Water (50 ml) was added to maltodextrin (25 gms) at 25-30° C. and stirred for 45 minutes at the same temperature. Sodium bromide (0.17 gms) was added to the reaction mixture at 25-30° C. Aqueous sodium hydroxide solution was added to the reaction mixture at 25-30° C. Sodium hypochlorite solution was added to the reaction mixture at 25-30° C. and stirred for 45 minutes at the same temperature. Methanol (150 ml) was slowly added to the reaction mixture at 25-30° C. and stirred for 45 minutes at the same temperature. Water (26.0 ml) was added to oxidized maltodextrin at 25-30° C. and stirred for 45 minutes at the same temperature. Ferric chloride (11.0 gms) was slowly added to a pre-cooled water (26.0 ml) at 10-15° C. and stirred for 15 minutes at the same temperature. Slowly added the ferric chloride solution to the reaction mixture at 25-30° C. Aqueous sodium carbonate solution was added to the reaction mixture at 25-30° C. and stirred for 30 minutes at the same temperature. Aqueous sodium hydroxide solution was added to the reaction mixture at 25-30° C. Heated the reaction mixture to 50-55° C. and stirred for 1 hour at the same temperature. Aqueous hydrochloric acid solution was added to the reaction mixture at 50-55° C. and stirred for 40 minutes at the same temperature. Heated the reaction mixture to 95-100° C. and stirred for 40 minutes at the same temperature. Cooled the reaction mixture to 25-30° C. Filtered the reaction mixture through hyflow bed. The obtained filtrate was purified by ultra-filtration technique. Acetone (260 ml) was added to the obtained compound at 25-30° C. and stirred for 1 hour at the same temperature. Filtered the precipitated solid, washed with acetone and dried to get the title compound.

Yield: 12.5 gms; Molecular Wt.: 182 kDa; Iron Content: 17.75%.

We claim:

1. A process for the preparation of iron (III) carboxymaltose, comprising
a) Oxidizing one or more maltodextrins with oxone,
b) reacting the oxidized maltodextrins with iron (III) salt to provide iron (III) carboxymaltose.

2. The process as claimed in claim 1 wherein, the mole ratio of oxone used is ranging from 1.0 to 5.0 moles per 1 mole of maltodextrin.

3. The process as claimed in claim 1 wherein, when one maltodextrins is present, the maltodextrins has a dextrose equivalent (DE) of between 5 and 20, and wherein, when a mixture of more than one maltodextrins is present, the dextrose equivalent of each individual maltodextrins is between 2 and 40, and the dextrose equivalent of the mixture is between 5 and 20.

4. The process as claimed in claim 1 wherein, the oxidation is carried out in an alkaline solution at a pH of about 8 to about 12.

5. The process as claimed in claim 1 wherein, oxidation is carried out at temperatures in the range of about 15° C. to about 40° C.

6. The process as claimed in claim 1 wherein, oxidation is carried out at temperatures in the range of about 25° C. to about 35° C.

7. The process as claimed in claim 1 comprising, oxidizing one or more maltodextrins in the presence of a catalyst is alkali bromides.

8. The process as claimed in claim 1 comprising oxidizing one or more maltodextrins in the presence of a catalyst is sodium bromide.

9. The process as claimed in claim 1, comprising isolating the oxidized maltodextrins.

10. The process as claimed in claim 9, dissolving the isolated oxidized maltodextrins and treating with iron (III) salt to provide iron (III) carboxymaltose.

11. The process as claimed in claims 1 & 8, comprising dia-filtering the oxidized maltodextrin.

12. The process as claimed in claim 1, comprising dia-filtering the iron (III) carboxymaltose.

13. The process as claimed in claim 12 comprises, isolating iron (III) carboxymaltose from the solution by precipitation.

14. The process as claimed in claim 13 wherein precipitation of iron (III) carboxymaltose is carried out by treating a solution containing iron (III) carboxymaltose with suitable solvent selected from ketone solvent, ester solvent or alcohol solvent.

15. Use of iron (III) carboxymaltose according to any of the preceding claims as a medicament in the treatment of iron deficiency anemia.

16. Use of iron (III) carboxymaltose according to any of the preceding claims as medicaments for parenteral administration.

17. Use of iron (III) carboxymaltose according to any of the preceding claims as a medicament for intravenous administration.
